(19) United States
(12) Patent Application Publication
(10) Pub. No.: US 2021/0137965 A1
(43) Pub. Date: May 13, 2021

(54) Application Of Xylan In The Preparation Of Drugs Or Food For Preventing Or Treating Osteoporosis

(71) Applicant: Guangxi Institute Of Botany, Chinese Academy Of Sciences, Guilin, Guangxi (CN)

(72) Inventors: Yuheng Zhou, Guilin, Guangxi (CN); Xiangxiang Qin, Guilin, Guangxi (CN); Aihua Cai, Guilin, Guangxi (CN); Haishan Chen, Guilin, Guangxi (CN); Yifang Lu, Guilin, Guangxi (CN); Ciyu Li, Guilin, Guangxi (CN); Hourui Zhang, Guilin, Guangxi (CN)

(73) Assignee: Guangxi Institute Of Botany, Chinese Academy Of Sciences, Guilin, Guangxi (CN)

(21) Appl. No.: 17/251,493

(22) PCT Filed: Oct. 11, 2018
(86) PCT No.: PCT/CN2018/109847

§ 371 Date: Jan. 6, 2021

(30) Foreign Application Priority Data
Jun. 11, 2018 (CN) 201810594637.6

(51) Int. Cl. A61K 31/717; A61P 19/10; A23L 33/125; A23L 33/00

(52) U.S. Cl. CPC A61K 31/717; A61P 19/10; A23V 2002/00; A23L 33/40; A23L 33/125

(57) ABSTRACT

The present disclosure relates to the prevention of osteoporosis and specifically relates to an application of xylan (polyxylose) in the preparation of drugs or food for preventing or treating osteoporosis. The present disclosure proves by utilizing high-purity xylan in animal experimentation that xylan significantly improves bone metabolism, increases blood vitamin D, PINP, and BALP levels, reduces blood CTXI and urine hydroxyproline levels, and reduces urinary calcium loss, thus providing the effects of inhibiting bone resorption and promoting bone formation. Compared with animals having cellulose, lignin, pectin, fructan, glucomannan intakes or having no dietary fiber intake, animals having a xylan intake have higher bone mass and bone density and better bone biomechanical properties. Xylan is suitable for further development into the drugs or food for preventing or treating osteoporosis.

This application claims priority to Chinese Patent Application No. 201810594637.6, entitled “APPLICATION OF XYLAN IN THE PREPARATION OF DRUGS OR FOOD FOR PREVENTING OR TREATING OSTEOPOROSIS”, filed to China National Intellectual Property Administration on Jun. 11, 2018, the entire contents of which are incorporated herein by reference.

### TECHNICAL FIELD

The present disclosure relates to the prevention of osteoporosis and specifically relates to an application of xylan in the preparation of drugs or food for preventing or treating osteoporosis.

### BACKGROUD

Osteoporosis (OP) is a systemic skeletal disorder characterized by a decrease in bone mass, degeneration of bone microstructure, thus resulting in increased brittleness of the bone, reduced bone mechanical strength, reduced load tolerance, and susceptibility to fracture. Osteoporosis can occur at any age, but the elderly are the main incidence group. According to incomplete statistics, the proportion of people over 60 years old suffering from osteoporosis in China has reached 59.89%. According to the aging process in China, it is predicted that by 2050, the elderly population aged over 60 years old will account for 20% of the total population in China. The population with osteoporosis will account for 13.2% of the total population. Osteoporosis has become a serious social health problem, and it is another disease that seriously endangers human health following tumors and cardiovascular diseases. It is of great social significance to take effective methods to prevent and treat osteoporosis.

There is no cure for osteoporosis. Currently, the preventive measures are mainly daily supplements of vitamin D and calcium, and the treatment measures are to use hormones, bisphosphonates and other drugs to inhibit the rapid bone loss of the elderly as well as parathyroid hormones, fluoride products and other drugs to promote the bone formation. The recent studies suggest that, supplements of calcium and vitamin D cannot reduce the incidence of fracture in middle-aged and elderly people over 50 years old, and a large supplementary dose of vitamin D also increase the risk of fracture. Therefore, it is very important to develop new safe and effective preparations to improve bone metabolism in the long run and to prevent or treat osteoporosis.

Although osteoporosis is the most common bone disease in middle-aged and elderly people, its generation is not only due to the imbalance of bone metabolism in elderly people, but also closely related to the accumulation of bone in the stages of adolescence and young adults. It is of great significance to maintain healthy bones for preventing osteoporosis in elderly people at all times of life. It has long been recognized that increasing dietary fiber intake can improve the health of bone. Chinese patent No.201380021560.9 disclosed an association compound of glucan from cereals with arabinoxylan, which is used as balanced intestinal microorganisms and used for the weight management of subjects suffering from constipation, inflammatory bowel syndrome, inflammatory bowel diseases, osteoporosis, and obesity, and used for preventing or treating cancers, especially colon cancer, diabetes, as well as conditions associated with oxidative stress and/or cardiovascular diseases. However, the patent does not explain any of its action mechanisms. A research literature in 2016 partially set forth the intestinal microbial mechanism of dietary fiber on bone health, that is, intestinal microorganisms as well as short-chain fatty acids generated from fermentation of dietary fiber by intestinal microorganisms improve the synthesis level of insulin-like growth factor-1 in organisms, thereby promoting the formation and growth of bone and improving the bone health. In other words, intestinal microorganisms are an important means to improve bone health. However, it is not clear what microorganisms play a major role in promoting bone health, and it is still not clear which type of dietary fiber is most effective in improving bone health.

In the study of correlation between osteoporosis and clinical biochemical indexes, it has been found that metabolic indexes such as estrogen level, urinary calcium level, VD level, PINP, BALP, CTX-I, urine hydroxyproline and other metabolic indicators are closely related to bone health, but there have not any reports so far about the association between dietary fiber and bone metabolism indexes. It is not clear whether dietary fiber can affect the levels of these indexes as well as which type of dietary fiber can positively regulate the levels of which indexes most effectively, thus significantly improving the bone health.

Dietary fiber in food is the general term of polysaccharides that cannot be digested by human digestive enzymes and can enter the large intestine for microbial fermentation and decomposition in the intestinal tract. There are a wide variety of dietary fibers, and those contained in human diet are mainly cellulose, hemicellulose and pectin. The three types of dietary fiber are the main constituents that make up the plant cell walls. Where, hemicellulose is the general term of a class of substances in the plant cell wall except cellulose and pectin, which are composed of more than two or three kinds of monoglycosyls, and have amorphous structure. The glycosyls that make up hemicellulose include D-xylosyl, D-mannosyl, D-glucosyl, D-galactosyl, L-arabinosyl, 4-O-methyl-D-glucuronyl, D-galacturonyl and D-glucuronyl, as well as a few L-rhamnose, L-fucose and the like. Hemicellulose is mainly classified into three types, i.e., polyxyloses (i.e., xylan), polyglucomannoses (i.e., glucomannan) and polygalactoglucomannoses.

Dietary fiber is the basic carbon source supporting the growth and fermentation of intestinal microorganisms, and the food components that affect the intestinal microecological structure and thus affect the normal physiological metabolism of human body. However, due to the different structures of different types of dietary fiber, a certain type of dietary fiber can only proliferate a specific type of intestinal microorganisms, and produce specific spectrum of metabolites, thus producing a positive regulatory effect to a specific metabolism aspect of the organism. Therefore, it is of great significance to identify the most effective component of basic food for improving the bone health but the intake of which is significantly deficient in daily dietary, then apply it to enhance the intake level of this component for modern people and thus improve the bone health levels of the public.

In the large family of dietary fiber, no dietary fiber is as closely related to the human diet as xylan. Xylan is the main component of hemicellulose, its content in the stems and spikes of graminaceous crops can reach 20-40%, and its content in the epidermis of cereals can also reach 15-50%. Since entering the agricultural society, as the role of food in the human diet, the xylan in the epidermis of cereals also accounted for more than 50% of the total dietary fiber for human. After the industrial civilization, the structure of human diet has changed dramatically. Food is not only high energy quantifiable, but the extensive processing of food results in the removal of xylan-rich epidermis, resulting in insufficient dietary fiber intake and the loss of xylan, the most important component in dietary fiber. The disturbance of intestinal flora caused by the imbalance of nutritional structure is the inducement of many modern diseases.

Due to the difficulties of extraction technology and commercial production of xylan, there has long been a lack of in-depth study of the physiological function of xylan. Xylan is only known as one of the components of dietary fiber, but its specific physiological function as an independent component has not been further learned more.

### SUMMARY

An objective of the present disclosure is to provide an application of xylan (polyxylose) in the preparation of drugs or food for preventing or treating osteoporosis. Xylan (polyxylose) can significantly improve bone metabolism, increase blood vitamin D, PINP, and BALP levels, reduce blood CTX-I and urine hydroxyproline levels, and reduce urinary calcium loss, thus providing the effects of inhibiting bone resorption, promoting bone formation, and preventing bone loss. Animals having a xylan intake have higher bone density (the gold standard for diagnosis of osteoporosis) and better bone mechanical properties (the most direct and visual indexes). Xylan (polyxylose) is suitable for further development into the drugs or food for preventing or treating osteoporosis.

To achieve the above objective, the present disclosure provides the following technical solution:

An application of xylan (polyxylose) in the preparation of drugs or food for preventing or treating osteoporosis.

Further, the xylan is applied in the preparation of drugs or food for enhancing bone mass, increasing bone mineral density, improving bone maximum load and improving bone resistance to fracture.

Further, the xylan is applied in the preparation of drugs or food for inhibiting bone resorption, enhancing bone formation, reducing bone loss, and improving bone metabolism.

Further, the xylan is applied in the preparation of drugs or food for reducing urinary calcium loss.

In the above applications of xylan in the preparation of drugs or food for preventing or treating osteoporosis, the xylan has the effects of reducing the level of a marker CTX-I in blood and reducing the level and the total daily discharge of hydroxyproline in blood and urine; the xylan has the effects of enhancing the levels of markers PINP and BALP in blood and increasing the level of vitamin D in blood.

In the above applications of xylan in the preparation of drugs or food for preventing or treating osteoporosis, the xylan can be used as an independent preparation, and also can be associated with other drugs to form a compound preparation, or associated with other food, thus to produce drugs or food for preventing or treating osteoporosis.

In the above applications of xylan in the preparation of drugs or food for preventing or treating osteoporosis, the xylan includes heteropolymeric xylan containing various side-chain groups and homopolymeric xylan without substituents, and the xylan refers to a polysaccharide polymer with a degree of polymerization greater than 10.

Further, the xylan refers to a chain backbone consisting of D-pyranoid xylose residues, as the constitutional units, linked by β-(1→4) glycosidic bonds or β-(1→3) glycosidic bonds, which is a class of polysaccharides with various unequal side-chain groups distributed at different positions in the chain backbone; the general structural formula is shown as below:

[chemical structure]

Wherein, R represents side-chain groups, including one or more of D-glucuronyl, 4-O-methyl-D-glucuronyl, D-glucosyl, L-arabinosyl, D-xylosyl, D or L galactosyl, rhamnosyl, acetyl, and feruloyl. For example:

Homogeneous xylan without substituents exists in a few plants. For example, xylan in green algae is linear homogeneous polyxylose linked by β-(1→3) glycosidic bonds; in some red algae, xylan is linear homogeneous polyxylose linked by β-(1→3) and β-(1→4) glycosidic bonds; and there are homogeneous xylan linked by β-(1→4) glycosidic bonds in Brachypodium pinnatum, tobacco stems, and Cyamopsis tetragonoloba husk.

The side-chain groups of hardwood xylan are mainly acetyl and 4-O-methyl-α-D-pyranoid glucuronyl. Acetyl is generally located at C3 position, 4-O-methyl-α-D-pyranoid glucuronyl is generally located at C2 position, and there is usually one 4-O-methyl-α-D-pyranoid glucuronic acid side chain for every 10 xylosyls, which is collectively called poly-O-acetyl-4-O-methylglucuronic acid xylose.

The side-chain groups of softwood xylan are mainly arabinosyl and 4-O-methylglucuronyl. α-L-furanoid arabinosyl is generally linked at C3 position of xylosyl in the chain backbone, while 4-O-methyl-α-D-glucuronyl is generally linked at C2 position, and there is usually one 4-O-methyl-α-D-glucuronyl side chain for every 5 to 6 xylosyls, which is collectively called polyarabinose-4-O-methylglucuronic acid xylose.

The side-chain groups of gramineous plant xylan are mainly L-furanoid arabinosyl, acetyl and 4-O-methyl-pyranoid glucuronyl. A typical form is that L-furanoid arabinosyl and 4-O-methyl-pyranoid glucuronyl are linked at positions C2 and C3 in the chain backbone of xylose respectively, and acetyl is linked at position C2 or C3, which is also called glucuronic acid arabinosyl xylan. In wheat, rye, barley, oat, corn, sorghum and other cereal endosperms as well as ryegrass and bamboo shoots, the xylan is mainly araboxylan, where C(O)-2 or C(O)-3 in the xylose residue of the chain backbone is mono-substituted by L-arabinosyl, or disubstituted at C(O)-2,3 positions simultaneously by L-arabinosyl.

Because of different plant sources, different plant parts, different extraction processes, and even different synthesis processes, the xylan may have different molecular polymerization degrees, different types of side-chain groups, and different substitution degrees, thereby finally showing difference in terms of molecular weight and structure.

In the applications of xylan in the preparation of drugs or food for preventing or treating osteoporosis, when xylan is utilized to prepare drugs or food for preventing or treating osteoporosis, it is added as a crude product or as extracts of various purities. The crude product mainly includes wheat bran, corn husk and various straw crushed materials with xylan as the physiological active component.

It is confirmed through studies in the present disclosure that, in the main types of dietary fiber in food naturally ingested by human, xylan is the most important component to improve bone health. In terms of improving clinical biochemical indexes related to osteoporosis and enhancing fracture resistance, xylan is significantly superior to other dietary fiber components and the control group without dietary fiber. Xylan has significant effects on both young and old animals, specifically including:

(1) Xylan can significantly increase the levels of bone formation markers, procollagen type I N-terminal peptide (PINP) and bone alkaline phosphatase (BALP), in the serum of rat. PINP and BALP are secreted by osteoblasts during the formation of bone tissue, which are specific indexes of osteoblast maturation and new bone formation. Xylan enhances the levels of these markers, suggesting that xylan can promote the activity of osteoblasts.

(2) Xylan reduces the levels of bone resorption markers, collagen type I carboxyl-terminal peptide (CTX-I) and hydroxyproline. CTX-I and hydroxyproline are one of products of bone tissue collagen type I broken down by osteoclasts, and are the most widely used and the most valuable markers reflecting the process bone resorption. Xylan reduces the levels of these markers, suggesting that xylan can significantly inhibit the decomposition of bone.

(3) Xylan enhances the absorption of vitamin D3. Vitamin D3 is an important hormone for regulating bone metabolism, which can promote the absorption of calcium and the increasing of bone density. Xylan enhances the level of this factor, suggesting that xylan can promote the absorption of bone calcium.

(4) Xylan can increase the bone mass. Osteoporosis is mainly characterized by reduced bone mass. The double regulation of xylan on bone formation and bone resorption results in an increase of bone mass, thus having an effect of anti-osteoporosis.

(5) Xylan can significantly reduce the urinary calcium loss. Urinary calcium loss is one of the important causes of bone mineral loss. Xylan significantly reduces the urinary calcium loss, which also indicates the inhibition on the bone resorption.

(6) Xylan can significantly increase the bone density. Bone density is an important index of bone strength, and also is the gold standard for the diagnosis of osteoporosis and the evaluation of therapeutic effects. It is examined by dual-energy X-ray absorptiometry that, the bone density of rats with xylan intake increases significantly, which can effectively prevent and improve osteoporosis.

(7) Xylan can enhance the biomechanical properties of bone. The increases of bone mass and bone density directly result in the enhancement of bone maximum load and fracture load, which are the main embodiments of the ultimate functions of xylan.

Compared with the prior art, substantial progresses achieved by the present disclosure are embodied in:

1. The present disclosure provides an application of xylan in the preparation of drugs or food for preventing or treating osteoporosis, thus providing new drugs or food for the treatment or prevention of osteoporosis.

2. By comparing the main types of dietary fiber ingested by human through studies, it is strongly proved in the present disclosure from the perspectives of bone density, bone biomechanical properties, and biochemical metabolism indexes that, the major anti-osteoporosis component in the main dietary fiber for human is xylan, and human intake of xylan helps to improve bone metabolism and form a good bone structure, thus enhancing the biomechanical properties of bone and preventing osteoporosis.

3. High-purity xylan is used in the experiments of the present disclosure, fully eliminating the interference of other fiber components and ensuring the conclusion of the present disclosure completely reliable, which are impossible from the studies of dietary fiber by using a low purity of xylan.

4. It is verified in both young and old-aged rats in the present disclosure that no matter young or old, xylan can help to form higher bone mass and bone density, and thereby achieving better bone mechanical properties; xylan has a good effect on improving bone health throughout the life, which was not found in previous studies.

5. It is also confirmed in the present disclosure that, compared with wheat bran containing natural xylan, the extracted xylan has a more significant effect on the improvement of bone, suggesting that extraction removes the hindrance of impurities and a loose structure is more beneficial to the fermentation of intestinal microorganisms, thus achieving greater bone health benefits with a smaller amount of xylan.

6. The present disclosure discloses an application of xylan in drugs and food for preventing and treating osteoporosis and its partial mechanism of action, providing guidance on how to build life-long bone health. The major food to provide an essential intake amount of xylan for human is coarse food grains that have not been refined. However, grain refinement has become the mainstream diet in modern society, so it is difficult for people to eat enough coarse food grains to meet the demand of xylan as the ancients did. To compensate for the lack of xylan in the diet, it is an important approach to add xylan to processed food. The processed food can include any of starches, dairy products, bean products, meat products, beverages, candies and biscuits. The addition of xylan in all the commercial food enables the food a healthcare function of preventing osteoporosis.

The same as other nutrient elements necessary to maintain normal physiological metabolism of people, xylan in the present disclosure is safe and non-toxic. A life-long sufficient intake of xylan is essential for fundamentally improving bone metabolism, building and maintaining optimal bone mass at all times of life, preventing and treating osteoporosis, which is used as a preventive nutrient and a therapeutic agent.

Xylan can be used as the principal component of a drug or used as an adjuvant to be associated with other anti-osteoporosis drugs to prepare a drug for preventing and resisting osteoporosis.

### DETAILED DESCRIPTION

The present disclosure will be further described in detail in combination with the detailed description below, but it should be understood that the following examples are intended to illustrate the present disclosure but not to limit its scope.

### EXAMPLE 1

### Effect of Xylan on Bone Mass of Middle-Aged and Elderly Female Rats

The level of estrogen in vivo has a great effect on the bone metabolism in women. After the middle-aged, with the gradual decline of the estrogen level, bone resorption tends to increase, bone loss is accelerated, and the risk of osteoporosis is significantly increased. This example is intended to study the effect of adding various dietary components in natural states on the bone metabolism of rats after child-bearing period, with the middle-aged female rats as the model animals. The added xylan, lignin, cellulose, pectin, and inulin represent the major components existed in the dietary fiber ingested by human. The mixed group is a mixture of xylan, cellulose, pectin, and inulin. Sesame is a natural anti-osteoporosis food according to folk traditions and as reported in documents. Chitosan oligosaccharide is a kind of saccharide with low molecular weight, soluble in water and naturally positive, which is generated from the degradation of chitosan. A large number of literatures reported that chitosan oligosaccharide has the effects of promoting osteogenesis and resisting osteoporosis. Xylan, lignin and cellulose are all prepared by the inventor, and the remaining are commercial products.

1. Materials

1.1 Test Animals

SFP-grade female SD rats (Hunan, SJA), at ages of 8 months, body weight 375±37 g, have completed multiple fertility tasks.

1.2 Xylan, Lignin and Cellulose

Preparation method of xylan: With bagasse as a raw material, the raw material was composted and fermented for 3 months to 1 year after spraying with tap water, washed with clear water to remove yellow water and impurities, then cooked at 100° C. with diluted alkaline at pH 12.0 for 2 h to remove acetyl and part of lignin. It was squeezed and then washed repeatedly. The residues were extracted with 8% (w/v) of NaOH solution at a solid-to-liquid ratio of 1:10 for 6 h at an extraction temperature of 80° C. and then separated the liquid from the solid. The liquid fraction was clarified on standing. The clarified liquid was separated over a membrane to separate small molecules less than 10000 Dalton out through the membrane, and the trapped fluid was dialyzed repeatedly by continuously adding clear water to remove the alkaline until about pH 12.0. The trapped fluid was bleached by adding a small amount of food-grade H₂O₂, then neutralized, then precipitated with 95% alcohol and washed repeatedly with 75% alcohol, until all the free lignin was washed off and the products became white. The resulting material was finally dried, wherein the content of xylosyl was 90% of the total mass.

The resulting xylan is membrane entrapment with a molecular weight cut off of 10000 Dalton molecular weight and is the product generated from precipitation with ethanol. It's a macromolecular substance, with an average molecular weight detected by nuclear magnetic resonance of 80000, the physical properties are as below: white emulsion when dissolved in water, insoluble in acid, soluble in alkaline, odorless, white or grey white, light yellow. The side-chain groups of xylan mainly include acetyl, arabinosyl, glucuronyl and 4-O-methyl-glucuronyl, wherein xylosyl:arabinosyl=9˜10:1.

Preparation method of cellulose: Bagasse was extracted with 10% (WN) of NaOH solution to get xylan and lignin. The solid and liquid were separated and the resulting residues were crude cellulose. The crude cellulose was extracted with 15% (W/V) of NaOH solution again at 100° C. for 12˜24 h, then the solid and liquid were separated again and the solid was washed to neutral, and finally cooked with 2% of H₂SO₄ (W/V) solution at 121° C. for 30 min. The remaining solid was washed to neutral, dried in an oven and crushed to get the cellulose with a purity >95%.

Preparation method of lignin: With bagasse as a raw material, xylan and lignin were extracted from the raw material using 10% (W/V) of NaOH solution as the extraction solvent. Xylan was entrapped from the solution by an ultrafiltration membrane with a molecular weight cut off of 100000 Dalton. The lignin solution in the filtrate was filtered again over a nano-filtration membrane with a molecular weight cut off of 1000 Dalton to remove the alkaline solution. The entrapped portion was nano-filtered by repeatedly adding pure water to near neutral, into which was then added acidic ethanol. The supernatant was separated by sedimentation and centrifugation, and then rectified to remove ethanol. The rectification residues were neutralized and spray-dried.

1.3 Drugs and Reagents

CUSABIO kit (Wuhan Huamei Biotech Co., Ltd.).

1.4 Instruments and Equipment

AG-201 Electronic Universal Testing Machine (Japan, Shimadzu Corporation); SP-Max 3500FL Multi-Mode Fluorescence Microplate Reader (Shanghai Flash Spectrum Biological Technology Co., Ltd.); HOLOGIC Discovery A dual-energy X-ray absorptiometry (US).

2. Experimental Methods

2.1 Groups and Administration

The purchased female rats were acclimated for 2 weeks, and then grouped into a blank control group (without dietary fiber), a xylan group, a lignin group, a cellulose group, a pectin group, an inulin group, a sesame group, a chitosan oligosaccharide group, and a mixed group, respectively. The formulation of basal feed was AIN-96M. The blank group was fed with the basal feed, and the remaining groups were fed with the basal feed together with 5% of test components. Feeding temperature was 25° C., humidity was 40-60%, free access to food and water, with a light cycle of 12 h/12 h.

2.2 Determination of Indexes

Before killing, urine was collected in a metabolism cage for 48 h, and the content of hydroxyproline in the urine was detected and a total discharge amount in 24 h was calculated.

When killing, rats were sacrificed by directly bleeding at the neck. Blood was collected and left to coagulate for 1 h at room temperature to precipitate the serum. After centrifugation at 3000 rpm for 10 min, serum was packaged and kept at −80° C. The markers of vitamin D, PINP, CTX-I, hydroxyproline and BALP in serum were determined with the CUSABIO kit following the instruction. After sacrificing the rats, the left and right femurs were stripped from the posterior limbs and weighed. The left femur was scanned with the dual-energy X-ray absorptiometry to determine the bone density. The right femur was subjected to a three point bending test on the Universal Testing Machine to determine the biomechanical properties of the right femur. Test conditions were: the span was 20 mm, the loading speed was 5 mm/min, a load-deformation curve was recorded, and the maximum load and other parameters were read from the curve.

2.3 Data Processing

A SPSS19.0 statistical software package was used for analysis. The calculation results were expressed as X±S. One-way ANOVA was used for comparison among groups, wherein difference P<0.05 was of statistical significance.

3 Experimental Results

3.1 Bone Mass

One feature of osteoporosis is the loss of bone mass. Compared with all other groups (as in

TABLE 1
Bone mass

| Groups | N | Weight of right femur (g) |
|---|---|---|
| Control | 10 (5 deaths) | 0.96 ± 0.39** |
| Xylan | 10 | 1.26 ± 0.09 |
| Cellulose | 10 (2 deaths) | 1.10 ± 0.09 |
| Lignin | 10 (2 deaths) | 1.25 ± 0.11 |
| Pectin | 10 | 1.13 ± 0.07 |
| Inulin | 10 | 1.00 ± 0.13* * |
| Sesame | 10 | 1.13 ± 0.06 |
| Chitosan oligosaccharide | 10 | 1.12 ± 0.06 |
| Mixed dietary fiber | 10 | 1.20 ± 0.14 |

Note:
Compared to xylan: *P < 0.05, **P < 0.01;

3.2 Level of Vitamin D

Vitamin D is an important hormone for regulating bone metabolism. At physiological doses, Vitamin D can promote the absorption of calcium in the intestinal tract, the reabsorption of calcium and phosphorus in renal tubules and the bone calcification, thus being beneficial to the increase of bone density. The levels of vitamin D in blood of xylan group are higher than those in other groups (except the sesame group), wherein the significance of difference compared to the control group and the chitosan oligosaccharide group is P<0.01, and the significance of difference compared to lignin group, pectin group, inulin group, and mixed dietary fiber group is P<0.05.

TABLE 2
Level of vitamin D in serum

| Groups | N | Vitamin D in blood (ng/ml) |
|---|---|---|
| Control | 10 | 32.57 ± 4.01** |
| Xylan | 10 | 39.15 ± 3.48 |
| Cellulose | 10 | 38.5 ± 3.9 |
| Lignin | 10 | 33.55 ± 5.51** |
| Pectin | 10 | 33.95 ± 4.77** |
| Inulin | 10 | 33.55 ± 3.66** |
| Sesame | 10 | 39.57 ± 2.97 |
| Chitosan oligosaccharide | 10 | 31.9 ± 4.51** |
| Mixed dietary fiber | 10 | 33.6 ± 5.69** |

Note:
Compared to xylan: *P < 0.05, **P < 0.01.

3.3 Level of PINP in Blood

Procollagen type I N-terminal peptide (PINP) is the N-terminal redundant peptide chains that are cut off when collagen is formed from procollagen type I. The content of PINP in serum indicates the ability of osteoblasts to synthesize ossein, which is a specific sensitivity index of new bone formation. As can be seen from Table 3 below, the level of PINP in the serum of xylan group is higher than those in other groups, the significance degree for pectin and chitosan oligosaccharide is P<0.01, and the significance degree for cellulose and inulin is P<0.05, suggesting that xylan has the effect of promoting osteogenesis.

TABLE 3
Level of PINP in blood

| Groups | N | PINP (pg/ml) |
|---|---|---|
| Control | 10 | 110.56 ± 11.77 |
| Xylan | 10 | 196.68 ± 62.58 |
| Cellulose | 10 | 56.22 ± 15.88** |
| Lignin | 10 | 104.18 ± 46.29 |
| Pectin | 10 | 30.34 ± 6.17** |
| Inulin | 10 | 75.20 ± 66.30** |
| Sesame | 10 | 143.70 ± 148.06 |
| Chitosan oligosaccharide | 10 | 41.28 ± 70.79** |
| Mixed dietary fiber | 10 | 101.06 ± 45.40 |

Compared to xylan: *P < 0.05, **P < 0.01.

3.4 Level of BALP in Blood

Bone-specific alkaline phosphatase (BALP) is an extracellular enzyme of osteoblasts, which is mainly used to hydrolyze inorganic phosphates and further reduce the concentration of pyrophosphates, thus being beneficial for osteogenesis. The activity of BALP is linearly related to the activities of osteoblasts and preosteoblasts. Therefore, it is considered to be the most precise bone formation marker, marking the maturation and activity of osteoblasts. The activity of BALP in the xylan group is higher than those in other groups, and obviously, xylan has the most excellent effect of osteogenesis.

TABLE 4
Level of BALP in blood

| Groups | N | Blood BALP (U/L) |
|---|---|---|
| Control | 10 | 79.9 ± 60.02 |
| Xylan | 10 | 96.36 ± 28.49 |
| Cellulose | 10 | 27.98 ± 36.2 |
| Lignin | 10 | 54.46 ± 48.35 |
| Pectin | 10 | 54.22 ± 51.80 |
| Inulin | 10 | 82.66 ± 94.84 |
| Sesame | 10 | 30.38 ± 36.11** |
| Chitosan oligosaccharide | 10 | 30.78 ± 28.11 |
| Mixed dietary fiber | 10 | 59.66 ± 50.01** |

Note:
Compared to xylan: *P < 0.05, **P < 0.01.

3.5 Level of CTX-I in Blood

Collagen type I carboxyl-terminal peptides (CTX-I) are short peptide fragments broken down from collagen type I, accounting for 90% of organic matters of bone, into the blood, which is the most widely used marker of collagen degradation. In Table 5 as below, except the sesame group, the level of CTX-I in serum of the xylan group is lower than those in other dietary fiber groups and that in the control group, suggesting that xylan has the effect of inhibiting bone resorption.

TABLE 5
Level of CTX-I in blood

| Groups | N | CTX-I (pg/ml) |
|---|---|---|
| Control | 10 | 955.21 ± 76.14 |
| Xylan | 10 | 953.45 ± 117.77 |
| Cellulose | 10 | 963.45 ± 117.87 |
| Lignin | 10 | 1026.55 ± 182.17 |
| Pectin | 10 | 1075.47 ± 156.89 |
| Inulin | 10 | 1045.68 ± 172.84 |
| Sesame | 10 | 827.64 ± 133.12 |
| Chitosan oligosaccharide | 10 | 990.55 ± 102.29 |
| Mixed dietary fiber | 10 | 1052.91 ± 202.36 |

Compared to xylan: *P < 0.05, **P < 0.01.

3.6 Level of Hydroxyproline in Blood and Urine

Hydroxyproline is a kind of amino acid in bone matrix. Hydroxyproline in blood and urine is the product generated from the breakdown of ossein, which is significantly related to the absorption rate of bone (Table 6 below). The level of hydroxyproline in blood and urine in the xylan group is significantly reduced compared to those in other groups, suggesting that xylan can inhibit the decomposition of bone.

TABLE 6
Level of hydroxyproline in blood and urine

| | | Level of | Hydroxyproline |
|---|---|---|---|
| | | hydroxyproline | in urine, 24 |
| Groups | N | in blood (μg/ml) | h (m) |
| Control | 10 | 53.58 ± 14.02* | 113.01 ± 32.16 |
| Xylan | 10 | 35.40 ± 6.37 | 70.61 ± 53.65 |
| Cellulose | 10 | 26.20 ± 5.11 | 84.91 ± 26.82 |
| Lignin | 10 | 38.76 ± 5.71 | 71.68 ± 40.19 |
| Pectin | 10 | 38.34 ± 4.18 | 195.43 ± 32.16** |
| Inulin | 10 | 37.26 ± 5.62 | 120.26 ± 56.95 |
| Sesame | 10 | 48.22 ± 14.15 | 267.11 ± 193.74** |
| Chitosan oligosaccharide | 10 | 49.98 ± 13.95 | 108.81 ± 20.85 |
| Mixed dietary fiber | 10 | 32.52 ± 13.19 | 227.91 ± 57.13** |

Note:
Compared to xylan: *P < 0.05, **P < 0.01.

3.7 Results of Bone Density

Bone density is the internationally recognized gold standard for measuring osteoporosis. It can be known from Table 7 that the bone density in the xylan group is higher than those in all the dietary fiber groups and the control group, suggesting that xylan is the most effective nutritional factor in the dietary fiber for preventing osteoporosis.

TABLE 7
Bone density

| | Number | Bone density of |
|---|---|---|
| Groups | of rats | left femur (g/cm²) |
| Control | 10 | 0.2617 ± 0.02** |
| Xylan | 10 | 0.2916 ± 0.018 |
| Cellulose | 10 | 0.2602 ± 0.00** |
| Lignin | 10 | 0.2823 ± 0.011 |
| Pectin | 10 | 0.2792 ± 0.013* |
| Inulin | 10 | 0.2870 ± 0.015 |
| Sesame | 10 | 0.2838 ± 0.009 |
| Chitosan oligosaccharide | 10 | 0.2633 ± 0.004** |
| Mixed dietary fiber | 10 | 0.2837 ± 0.013 |

Note:
Compared to xylan: *P < 0.05, **P < 0.01.

3.8 Results of Three Point Bending Test

The function of bone is mainly to meet the biomechanical requirements of the body and to protect and support the body. The most important index in the three point bending test is maximum load, which reflects the intrinsic quality of the bone and is independent of the size of the bone. As can be seen from Table 8 below, xylan has higher maximum load and better resistance to fracture compared with other kinds of dietary fiber and the control group. Moreover, in terms of trend, the results of maximum load in the three point bending test (Table 8 below) correspond with the bone density, and higher bone density enables higher resistance to fracture.

TABLE 8
Results of three point bending test

| | Number | Maximum load of right |
|---|---|---|
| Groups | of rats | femur (Newton) |
| Control | 10 | 142.03 ± 31.52** |
| Xylan | 10 | 174.50 ± 27.53 |
| Cellulose | 10 | 122.80 ± 8.42** |
| Lignin | 10 | 154.04 ± 19.87* |
| Pectin | 10 | 148.29 ± 20.74* |
| Inulin | 10 | 160.96 ± 20.95 |
| Sesame | 10 | 155.27 ± 21.76* |
| Chitosan oligosaccharide | 10 | 140.04 ± 18.87** |
| Mixed dietary fiber | 10 | 160.44 ± 32.21 |

Note:
Compared to xylan: *P < 0.05, **P < 0.01.

4. Conclusion

From the view of combined data, xylan showed excellent overall performance no matter in terms of bone weight, the level of vitamin D in blood, or the levels of osteogenic and osteoclastic markers in blood or urine, as well as the bone density as a result of bone metabolism accumulation and ultimately the actual resistance to fracture, suggesting that the most effective anti-osteoporosis component in the main dietary fiber or dietary components for human is xylan, and the most effective diet or dietary fiber component for preventing senile osteoporosis is also xylan.

### EXAMPLE 2

### Effect of Xylan on the Bone of Rats During the Growing Period

Dietary fiber in food is the general term of polysaccharides that cannot be digested by human digestive enzymes, but can be degraded by enzymes secreted by intestinal microorganisms in the gut (mainly the large intestine) into small molecules that are available to microorganisms. The main components of dietary fiber include cellulose, xylan, pectin, fructan or glucan, and mannan. The objective of this example is to verify the effects of different dietary fiber components or combination thereof on the bone growth of rats during the growing period. Where, cellulose and xylan are prepared by the inventor, the purity of cellulose is ≥95%, and the purity of xylan is ≥85%; and pectin, inulin, Konjac gum, and wheat bran are all purchased from commercial production companies. The main component of inulin is fructan; the main component of Konjac gum is mannan; and wheat bran is a natural mixture of xylan, cellulose, fructan, and mannan, wherein xylan accounts for more than 50% of the total wheat bran; and the mixed dietary fiber group is obtained by mixing xylan, cellulose, fructan, and mannan.

1. Materials

1.1 Test Animals

SPF-grade male rats (Hunan, SJA), at ages of 2 months, body weight of about 200±20 g.

1.2 Xylan and Cellulose

Preparation method of xylan: With corncob as a raw material, the raw material was composted and softened, washed with clear water to remove impurities, then extracted with 8% (w/v) of NaOH solution at a solid-to-liquid ratio of 1:8 for 12 h at an extraction temperature of 80° C. and squeezed to separate the liquid from the solid. The liquid fraction was clarified on standing. The clarified liquid was separated over a membrane to separate small molecules less than 10000 Dalton out through the membrane, and the trapped fluid was dialyzed repeatedly to remove alkaline by adding clear water until about pH 12.0. The trapped fluid was bleached by adding a small amount of food-grade H₂O₂, then neutralized and precipitated with ethanol, then the precipitate was washed repeatedly with 75% alcohol until all the free lignin was washed off, and the resulting material was finally dried, wherein the product pentosyl accounted for 85% of the total mass. The main side-chain groups of the prepared xylan are acetyl, arabinosyl, glucuronyl, and 4-O-methylglucuronyl, wherein xylosyl:arabinosyl=7-9:1. Preparation method of cellulose: bagasse was extracted with 10% (W/V) NaOH for 6-24 h, xylan and lignin were dissolved, the solid and liquid were separated by squeezing or centrifugation, and the residues were washed. The resulting residues were soaked in 15% (W/V) NaOH solution again at 100° C. for 12 h, and subjected to solid-liquid separation again. The solid was washed with clear water to neutral, dried and then cooked with 2% (W/V) H₂SO₄ solution at 121° C. for 30 min to completely remove xylan. After cooking, the cellulose residues were washed to neutral, dried and crushed to get the cellulose with a purity>95%.

1.3 Drugs and Reagents

CUSABIO kit (Wuhan Huamei Biotech Co., Ltd.).

1.4 Instruments and Equipment

AG-201 Electronic Universal Testing Machine (Japan, Shimadzu Corporation);

NOVAA400P Atomic Absorption Spectrometer (Analytik Jena AG, Germany)

2. Experimental Methods

2.1 Groups and Administration

70 SPF-grade male rats were purchased and acclimated for 2 weeks, and then randomly grouped into 7 groups, 10 rats per group. The formulation of basal feed was AIN-96M. Each group was fed with the basal feed together with 5% of xylan, pectin, cellulose, Konjac, inulin, wheat bran, and dietary fiber combination respectively. Feeding temperature for rats was 25° C., humidity was 40-60%, free access to food and water, with a light cycle of 12 h/12 h.

2.2 Determination of Indexes

Rats were fed to an age of 12 months. Before killing, urine was collected in a metabolism cage for 48 h, and the urinary calcium was determined by flame atomic absorption spectrometry and a total discharge amount of urinary calcium in 24 h was calculated. When killing, rats were sacrificed by bleeding at the neck. Blood was collected and left for more than 30 min, then the serum was separated by low speed centrifugation and various indexes were determined. The right femur was stripped and weighed, and then subjected to a three point bending test on the

Universal Testing Machine to determine the biomechanical properties of the right femur. Test conditions were: the span was 20 mm, the loading speed was 5 mm/min, a load-deformation curve was recorded, and the maximum load and other parameters were read from the curve. The left femur was scanned with the dual-energy X-ray absorptiometry to determine the bone density.

2.3 Data Processing

A SPSS19.0 statistical software package was used for analysis. The calculation results were expressed as X±S. One-way ANOVA was used for comparison among groups, wherein difference P<0.05 was of statistical significance.

3. Experimental Results

3.1 Discharge Amount of Urinary Calcium in 24 h

Calcium is the main component of bone. High urinary calcium is closely related to loss of bone mass and osteoporosis. Investigation of urinary calcium excretion may reflect the loss of bone mass. As can be seen from Table 9 below, xylan has a significantly reduced discharge amount of urinary calcium in 24 h compared with the dietary fibers that are the most commonly ingested in the diet of human or their mixed combination, with P<0.01.

TABLE 9
Discharge amount of urinary calcium in 24 h

| | | Discharge amount of urinary |
|---|---|---|
| Groups | N | calcium in 24 h (mg) |
| Xylan | 10 | 0.83 ± 0.41 |
| Cellulose | 10 | 2.67 ± 2.57 |
| Pectin | 10 | 55.46 ± 22.80** |
| Inulin | 10 | 19.97 ± 9.20** |
| Konjac | 10 | 11.16 ± 8.96** |
| Wheat bran | 10 | 14.19 ± 7.67** |
| Mixed group | 10 | 13.95 ± 10.83** |

Note:
Compared to xylan: *P < 0.05, **P < 0.01.

3.2 Bone Weight

Senile osteoporosis is not only due to an excessive rate of bone loss in old age, but also closely related to the peak bone mass established during the growth period. As can be seen from Table 10 below, the test animals at the growth period can get higher bone mass through xylan diet, and it is obvious that long-term ingestion of xylan during the juvenile period is helpful to prevent senile osteoporosis.

TABLE 10
Bone weight

| Groups | N | Weight of left femur (g) |
|---|---|---|
| Xylan | 10 | 1.684 ± 0.117 |
| Cellulose | 10 | 1.60 ± 0.119 |
| Pectin | 10 | 1.486 ± 0.142** |
| Inulin | 10 | 1.68 ± 0.143 |
| Konjac | 10 | 1.54 ± 0.145* |
| Wheat bran | 10 | 1.56 ± 0.105* |
| Mixed group | 10 | 1.62 ± 0.07 |

Note:
Compared to xylan: *P < 0.05, **P < 0.01.

3.3 Results of Three Point Bending Test

As can be seen from Table 11 below, the xylan group has a higher maximum load compared with other kinds of dietary fibers, suggesting that ingestion of xylan during the growth period directly results in the enhancement of biomechanical properties of bone.

TABLE 11
Results of three point bending test

| | | Maximum load of |
|---|---|---|
| Groups | N | right femur (Newton) |
| Xylan | 10 | 212.16 ± 27.83 |
| Cellulose | 10 | 188.68 ± 43.91 |
| Pectin | 10 | 154.03 ± 29.10** |
| Inulin | 10 | 176.87 ± 29.01 |
| Konjac | 10 | 174.13 ± 41.45 |
| Wheat bran | 10 | 183.66 ± 17.27 |
| Mixed group | 10 | 178.75 ± 35.52 |

Note:
Compared to xylan: *P < 0.05, **P < 0.01.

4. Conclusion

The peak bone growth in rats is before 12 months of age. Compared with ingestion of any other single dietary fiber or some mixed fibers, long-term ingestion of xylan during the growth period results in better biomechanical indexes and better resistance to fracture. Xylan helps to promote the bone growth and the establishment of bone peak of animals during the growth period, thus effectively preventing senile osteoporosis.

### EXAMPLE 3

### Effect of Xylan on the Bone Density of Ovariectomized Rats with Osteoporosis

Postmenopausal osteoporosis is the most common chronic disease among middle-aged and elderly women. In this example, ovariectomized rats were used to simulate postmenopausal women to examine the effects of xylan and various dietary fibers on the bone of ovariectomized rats.

1. Test Animals

80 SFP-grade female SD rats (Hunan, SJA), at ages of 8 months, body weight 375±37 g, have completed multiple fertility tasks.

2. Preparation of Osteoporosis Rat Models

Osteoporosis rat models were established with bilateral ovariectomized rats. The rat models were anaesthetized with 10% chloral hydrate injection (3 ml/Kg body weight). Longitudinal incisions were made on both sides of the spine in the lower back to expose ovarian tissue. After ligating the surrounding tissue with silk thread, the mulberry-like ovary was completely removed. The wounds were sutured layer by layer. For the sham-operative group, the bilateral ovaries were exposed in the same way but not ectomized, and then injected with penicillin at 40,000 U per rat for three days to prevent infection.

3. Groups and Administration

The rats were divided into a nonoperative group, a sham-operative group, an operative blank group, and an operative administration group, with 20 rats per group. The formulation of basal feed was AIN-96M. The operative administration group was fed with the basal feed together with 5% of xylan (xylan: derived from bagasse, its side-chain groups mainly include acetyl, arabinosyl, glucuronyl and 4-O-methyl-glucuronyl, where xylosyl:arabinosyl=10˜15:1.), and all the other groups were only fed with the basal feed. During the experiments, the rats had free access to food and water.

4. Determination of Indexes5

After being fed for 4 months, the rats were fasted and sacrificed. Bilateral femurs were stripped from the posterior limbs, wrapped with gauze soaked in normal saline, and kept in a refrigerator at −80° C. During determination, the femurs were thawed naturally to room temperature. The biomechanical properties of right femur were determined by a three point bending test on the Universal Testing Machine. Test conditions were: the span was 20 mm, the loading speed was 5 mm/min, a load-deformation curve was recorded, and the maximum load and other parameters were read from the curve. The left femur was scanned with the dual-energy X-ray absorptiometry to determine the bone density.

5. Data Processing

A SPSS19.0 statistical software package was used for analysis. The calculation results were expressed as X±S. One-way ANOVA was used for comparison among groups, wherein P<0.05 was taken as the criteria of significant difference.

6. Experimental Results

As shown in Table 12. 4 months after removing the ovary, the bone density in the operative control group was lower than those in the nonoperative group and the sham-operative group, and the differences were of statistical significance, suggesting the successful establishment of osteoporosis models. The bone density in the operative administration group is significantly higher than that in the operative control group, suggesting that the bone loss caused by ovariectomy is inhibited by xylan.

TABLE 12
Bone density and the results of three point bending test

| | | Bone density | Maximum |
|---|---|---|---|
| Groups | N | (g/CM²) | load (Newton) |
| Nonoperative group | 20 | 0.2838 ± 0.009## | 161.21 ± 10.05## |
| Sham-operative | 20 | 0.2802 ± 0.012## | 160.96 ± 8.14## |
| group | | | |
| Operative | 20 | 0.2602 ± 0.007** | 141.68 ± 14.53** |
| control group | | | |
| Operative | 20 | 0.2829 ± 0.016## | 159.10 ± 15.52# |
| administration | | | |
| group | | | |

Note:
Compared to the nonoperative group, *P <0.05, **P <0.01; compared to the operative control group, #PP < 0.05, ##P < 0.01.

7. Conclusion

Xylan can inhibit the bone loss in ovariectomized rats, thus having the effect of preventing osteoporosis in female rats.

### EXAMPLE 4

The xylan in the present disclosure refers to a chain backbone consisting of D-pyranoid xylose residues, as the constitutional units, linked by β-(1→4) glycosidic bonds or β-(1→3) glycosidic bonds, which is a class of polysaccharides with various unequal side-chain groups distributed at different positions in the chain backbone. Where, the preparation method of xylan with corn husk as the raw material is as below: Corn husk was used as the raw material, and washed with clear water to remove impurities, then extracted with 8% (w/v) of NaOH solution at a solid-to-liquid ratio of 1:8 for 12 h at an extraction temperature of 80° C. and squeezed to separate the liquid from the solid. The liquid fraction was clarified on standing. The clarified liquid was separated over a membrane to separate small molecules less than 10000 Dalton out through the membrane, and the trapped fluid was dialyzed repeatedly to remove alkaline by adding clear water until about pH 12.0. The trapped fluid was bleached by adding a small amount of food-grade H₂O₂, then neutralized and precipitated with ethanol, then the precipitate was washed repeatedly with 75% alcohol until all the free lignin was washed off, and the resulting material was finally dried. The main side-chain groups of the prepared xylan are acetyl, arabinosyl, glucuronyl, and 4-O-methylglucuronyl, wherein xylosyl:arabinosyl=1˜2:1.

### EXAMPLE 5

The xylan in the present disclosure refers to a chain backbone consisting of D-pyranoid xylose residues, as the constitutional units, linked by β-(1→4) glycosidic bonds or β-(1→3) glycosidic bonds, which is a class of polysaccharides with various unequal side-chain groups distributed at different positions in the chain backbone. Where, the preparation method of xylan with wheat bran as the raw material is as below: Wheat bran was used as the raw material, and washed with clear water to remove impurities, then extracted with 8% (w/v) of NaOH solution at a solid-to-liquid ratio of 1:9 for 10 h at an extraction temperature of 80° C. and squeezed to separate the liquid from the solid. The liquid fraction was clarified on standing. The clarified liquid was separated over a membrane to separate small molecules less than 10000 Dalton out through the membrane, and the trapped fluid was dialyzed repeatedly to remove alkaline by adding clear water until about pH 12.0. The trapped fluid was bleached by adding a small amount of food-grade H₂O₂, then neutralized and precipitated with ethanol, then the precipitate was washed repeatedly with 75% alcohol until all the free lignin was washed off, and the resulting material was finally dried. The main side-chain groups of the prepared xylan are acetyl, arabinosyl, glucuronyl, and 4-O-methylglucuronyl, wherein xylosyl:arabinosyl=1˜3:1.

The description of the above examples is only intended to assist in understanding the method and core concept of the present disclosure. It should be noted that several improvements and modifications can be made to the present disclosure by persons with ordinary skills in the art without deviating from the principle of the present disclosure, all of which also fall within the protection scope of claims of the present disclosure. Various modifications to these examples are apparent to technical personnel in the art. General principles defined herein can be realized in other examples without deviating from the spirit or scope of the present disclosure. Therefore, the present disclosure shall not be confined to these examples set forth herein, but shall conform to the widest scope consistent with the principle and novel features disclosed herein.

1. An application of xylan in the preparation of drugs or food for preventing or treating osteoporosis.

2. The application of xylan in the preparation of drugs or food for preventing or treating osteoporosis according to claim 1, wherein: the xylan is applied in the preparation of drugs or food for enhancing bone mass, increasing bone mineral density, improving bone maximum load and improving bone resistance to fracture.

3. The application of xylan in the preparation of drugs or food for preventing or treating osteoporosis according to claim 1, wherein: the xylan is applied in the preparation of drugs or food for inhibiting bone resorption, enhancing bone formation, reducing bone loss, and improving bone metabolism.

4. The application of xylan in the preparation of drugs or food for preventing or treating osteoporosis according to claim 1, wherein: the xylan is applied in the preparation of drugs or food for reducing urinary calcium loss.

5. The application of xylan in the preparation of drugs or food for preventing or treating osteoporosis according to claim 1, wherein: the xylan has the effects of reducing the level of a marker CTXI in blood and reducing the level and the total daily discharge of hydroxyproline in blood and urine.

6. The application of xylan in the preparation of drugs or food for preventing or treating osteoporosis according to claim 1, wherein: the xylan has the effects of enhancing the levels of markers BALP and PINP in blood and increasing the level of vitamin D in blood.

7. The application of xylan in the preparation of drugs or food for preventing or treating osteoporosis according to claim 1, wherein: the xylan can be used as an independent preparation, and also can be associated with other drugs to form a compound preparation, or associated with other food, thus to produce drugs or food for preventing or treating osteoporosis.

8. The application of xylan in the preparation of drugs or food for preventing or treating osteoporosis according to claim 1, wherein: the xylan comprises heteropolymeric xylan containing various side-chain groups and homopolymeric xylan without substituents, and the xylan refers to a polysaccharide polymer with a degree of polymerization greater than 10.

9. The application of xylan in the preparation of drugs or food for preventing or treating osteoporosis according to claim 1, wherein: the xylan refers to a chain backbone consisting of xylose residues, as the constitutional units, linked by β-(1→4) glycosidic bonds or β-(1→3) glycosidic bonds, which is the general term of a class of polysaccharides with various different side-chain groups generally distributed at different positions in the chain backbone; the general structural formula is shown as below: [chemical structure]
Wherein, R represents side-chain groups, including one or more of D-glucuronyl, 4-O-methyl-D-glucuronyl, D-glucosyl, L-arabinosyl, D-xylosyl, D or L galactosyl, rhamnosyl, acetyl, and feruloyl;
The xylan can be derived from naturally occurring forms, or derived from some variations generated from a certain production process and having some changes in the natural structure, or derived from some variations synthesized by a certain process; the above xylans of different sources exhibit differences in terms of structure and molecular weight due to different degrees of polymerization, different degrees of substitution of side-chain groups and different types of substituents.

10. The application of xylan in the preparation of drugs or food for preventing or treating osteoporosis according to claim 1, wherein: when xylan is utilized to prepare drugs or food for preventing or treating osteoporosis, it is added as a crude product or as extracts of various purities; the crude product comprises wheat bran, corn husk and various straw crushed materials with xylan as the physiological active component.

11. The application of xylan in the preparation of drugs or food for preventing or treating osteoporosis according to claim 2, wherein: the xylan has the effects of reducing the level of a marker CTXI in blood and reducing the level and the total daily discharge of hydroxyproline in blood and urine.

12. The application of xylan in the preparation of drugs or food for preventing or treating osteoporosis according to claim 3, wherein: the xylan has the effects of reducing the level of a marker CTXI in blood and reducing the level and the total daily discharge of hydroxyproline in blood and urine.

13. The application of xylan in the preparation of drugs or food for preventing or treating osteoporosis according to claim 4, wherein: the xylan has the effects of reducing the level of a marker CTXI in blood and reducing the level and the total daily discharge of hydroxyproline in blood and urine.

14. The application of xylan in the preparation of drugs or food for preventing or treating osteoporosis according to claim 2, wherein: the xylan has the effects of enhancing the levels of markers BALP and PINP in blood and increasing the level of vitamin D in blood.

15. The application of xylan in the preparation of drugs or food for preventing or treating osteoporosis according to claim 3, wherein: the xylan has the effects of enhancing the levels of markers BALP and PINP in blood and increasing the level of vitamin D in blood.

16. The application of xylan in the preparation of drugs or food for preventing or treating osteoporosis according to claim 4, wherein: the xylan has the effects of enhancing the levels of markers BALP and PINP in blood and increasing the level of vitamin D in blood.
